(19) United States
(12) Patent Application Publication
(10) Pub. No.: US 2005/0265935 A1
(43) Pub. Date: Dec. 1, 2005

(54) Semiconductor nanocrystal quantum dots and metallic nanocrystals as UV blockers and colorants for suncreens and/or sunless tanning compositions

(76) Inventors: Jennifer A. Hollingsworth, Los Alamos, NM (US); Victor I. Klimov, Los Alamos, NM (US); Polina Olegovna Anikeeva, Los Alamos, NM (US)

Correspondence Address: UNIVERSITY OF CALIFORNIA;LOS ALAMOS NATIONAL LABORATORY, P.O. BOX 1663, MS A187, LOS ALAMOS, NM, 87545, US

(21) Appl. No.: 10/857,583

(22) Filed: May 28, 2004

(51) Int. Cl. A61K 7/42

(52) U.S. Cl. 424/59

(57) ABSTRACT

The present invention is directed to photostable sunscreen and/or artificial tanning compositions including quantum dot nanocrystals of a material selected from semiconductor nanocrystals, modified semiconductor nanocrystals, multicomponent semiconductor/semiconductor nanocrystals, and hybrid semiconductor/metal nanocrystals, the quantum dot nanocrystals having an absorption band gap occurring at wavelengths higher than 400 nm whereby the quantum dot nanocrystals have substantial broadband absorption properties of ultraviolet light at wavelengths across the range of both UV-A (320-400 nm) and UV-B (280-320 nm), and a dermatologically acceptable carrier for the quantum dot nanocrystals. The present invention is further directed to photostable sunscreen and/or artificial tanning compositions including a material selected from metallic nanocrystals, multicomponent metal/metal nanocrystals, and alloyed metal nanocrystals, the metallic material having a surface plasmon resonance occurring sufficiently into the visible or infrared spectral region whereby broad absorption features due to electronic transitions, the broad absorption features located at higher energies, provide substantial broadband absorption properties of ultraviolet light at wavelengths across the range of both UV-A (320-400 nm) and UV-B (280-320 nm), and a dermatologically acceptable carrier for the metallic material.

### STATEMENT REGARDING FEDERAL RIGHTS

This invention was made with government support under Contract No. W-7405-ENG-36 awarded by the U.S. Department of Energy. The government has certain rights in the invention.

### FIELD OF THE INVENTION

The present invention relates to sunscreen compositions and sunless tanning compositions, especially such sunscreen compositions and such sunless tanning compositions wherein they include selected semiconductor nanocrystal quantum dots, metallic nanocrystals, modified-semiconductor nanocrystal quantum dots, multicomponent semiconductor/semiconductor nanocrystals, and/or hybrid semiconductor/metal nanocrystals.

### BACKGROUND OF THE INVENTION

Exposure of human skin to sunlight imparts a tan to the skin. However, such exposure has significant health risks including sunburn, as well as the development of melanomas and other forms of skin cancer. Prolonged exposure to sunlight can also accelerate the natural aging process in the skin. In addition, ever increasing concerns have been raised about the need to provide both UV-A and UV-B protection as each has known or suspected health risks. For example, UVA rays penetrate more deeply into the skin and can cause long-term damage, while UVB rays are the primary cause of sunburn. As a result, dermatologists encourage individuals to use sunscreen compositions for protection from the sun.

Sunscreen compositions can provide protection from UVB radiation (280-320 nm), UVA radiation (320-400 nm), or both. One type of sunscreen composition employs physical screening agents, e.g., particles of transition metal oxides such as titanium oxide or zinc oxide. These particles have sometimes been applied in the form of thick, opaque creams that diffuse and scatter UV radiation. Where the particle sizes have been large (generally above about 0.1 micron), the resulting opacity (“whitening”) diminishes aesthetic appeal. More recently, metal oxides such as titanium dioxide and zinc oxide have been applied in the form of smaller particles (from about 40 to 50 nanometers to avoid the whitening effect). There are numerous commercial sunscreen compositions employing such “transparent” metal oxides.

A drawback to the use of titanium dioxide and zinc oxide compositions is that they are relatively transparent in the far UVA spectral region, i.e., from about 380 to 400 nm (see, e.g., FIG. 28 in U.S. Published application 2003/0161795). Further, there have been some questions raised regarding health and safety issues with the metal oxides, i.e., titanium dioxide and zinc oxide, commonly found in sunscreen compositions (see, e.g., Dunford et al., Chemical oxidation and DNA damage catalyzed by inorganic sunscreen ingredients, FEBS Letters, 418 (1997), 87-90).

Other numerous commercial sunscreen compositions employ organic compounds as sunscreen agents, e.g., octyl methoxycinnamate (OMC), 4-methylbenzylidene camphor (4-MBC), avobenzone, oxybenzone and homosalate. The organic compounds primarily work by absorbing UV light and dissipating the light as heat. Such organic compounds are often used in combination in order to provide some broadband effectiveness, i.e., some effectiveness in both the UVA and UVB regions. Yet, many organic sunscreen agents typically chemically degrade in sunlight and lose their effectiveness. There have also been questions raised regarding organic chemical sunscreens and DNA photodamage they may cause (see, e.g., Inbaraj et al., Photochemistry and Photobiology, 75 (2002), 107-116).

Thus, there remains a need for sunscreen compositions that can provide broadband protection across the entire UVA and UVB regions without degradation from the sunlight.

Some individuals desire the appearance of a tan without exposure to the sun by use of sunless tanning compositions. Sunless tanning compositions are typically based upon organic compounds such as α-hydroxy ketones, such as dihydroxyacetone (DHA), and α-hydroxyaldehydes. A chemical reaction between these organic compounds and components of the skin provides the artificial tanning effect. Yet, sunless tanning compositions do not always provide UVA protection and UVB protection and when they do, they suffer the same drawbacks as the sunscreen compositions.

Accordingly, there is a need for sunless tanning compositions that can provide the desired tanned look while providing broadband protection across the entire UVA and UVB regions without degradation from the sunlight.

It is an object of the present invention to provide a sunscreen composition and/or a sunless tanning composition that is truly broadband, that is, having effective protection from the sun in both the entire UVA region and the entire UVB region of the spectrum.

It is a further object of the invention to provide a sunscreen composition that is photostable and visibly transparent.

It is a still another object of the invention to provide a sunscreen composition also useful as a sunless tanning composition, such a sunscreen composition having effective protection from the sun in both the UVA region and the UVB region of the spectrum and yielding a desirable tan-like appearance.

### SUMMARY OF THE INVENTION

In accordance with the purposes of the present invention, as embodied and broadly described herein, the present invention provides a photostable sunscreen and/or artificial tanning composition including an effective amount of quantum dot nanocrystals of a material selected from the group consisting of non-oxide semiconductor nanocrystals, modified semiconductor nanocrystals, multicomponent semiconductor/semiconductor nanocrystals, and hybrid semiconductor/metal nanocrystals, the quantum dot nanocrystals having an absorption band gap occurring at wavelengths higher than 400 nm whereby the quantum dot nanocrystals have substantial broadband absorption properties of ultraviolet light at wavelengths across the range of both UV-A (320-400 nm) and UV-B (280-320 nm), and, a dermatologically acceptable carrier for the quantum dot nanocrystals. In one embodiment, metallic nanocrystals can be further included in the photostable sunscreen and/or artificial tanning composition.

The present invention further provides a photostable sunscreen and/or artificial tanning composition having broadband absorption properties within the range of both UV-A (320-400 nm) and UV-B (280-320 nm) including an effective amount of metallic nanocrystals having a surface plasmon resonance occurring sufficiently into the visible or infrared spectral region whereby broad absorption features due to electronic transitions, the broad absorption features located at higher energies, provide substantial broadband absorption properties of ultraviolet light at wavelengths across the range of both UV-A (320-400 nm) and UV-B (280-320 nm), and, a dermatologically acceptable carrier for the metallic nanocrystals.

The present invention further provides photostable sunscreen and/or artificial tanning composition having broadband absorption properties within the range of both UV-A (320-400 nm) and UV-B (280-320 nm) including an effective amount of nanocrystal quantum dot pigments of iron oxide, the iron oxide nanocrystal quantum dot pigments having an absorption band gap occurring at wavelengths higher than 400 nm whereby the iron oxide nanocrystal quantum dot pigments have substantial broadband absorption properties of ultraviolet light at wavelengths across the range of both UV-A (320-400 nm) and UV-B (280-320 nm), the iron oxide nanocrystal quantum dot pigments characterized as having at least one monodispersed size distribution and the iron oxide nanocrystal quantum dot pigments also characterized as soluble by ligand-stabilization of the iron oxide nanocrystal quantum dot pigments, and, a dermatologically acceptable carrier for the nanocrystal quantum dot pigments.

The present invention still further provides a process of protecting against detrimental effects of ultraviolet light at wavelengths across the range of both UV-A (320-400 nm) and UV-B (280-320 nm) by applying a photostable sunscreen composition including: (i) nanocrystals of a material from the group of semiconductor nanocrystal quantum dots, modified semiconductor nanocrystal quantum dots, metallic nanocrystals, and hybrid semiconductor/metal nanocrystals, the semiconductor nanocrystals having an absorption band gap occurring at wavelengths higher than 400 nm whereby the semiconductor nanocrystals have substantial broadband absorption properties of ultraviolet light at wavelengths across the range of both UV-A (320-400 nm) and UV-B (280-320 nm) and the metallic nanocrystals having a surface plasmon resonance occurring sufficiently into the visible or infrared spectral region whereby broad absorption features due to electronic transitions, the broad absorption features located at higher energies, provide substantial broadband absorption properties of ultraviolet light at wavelengths across the range of both UV-A (320-400 nm) and UV-B (280-320 nm); and, (ii) a dermatologically acceptable carrier for the nanocrystals.

### BRIEF DESCRIPTION OF THE DRAWINGS

FIG. 1 shows a plot of calculated sun protection factor (SPF) for a commercial sunscreen and three separate blends including the same commercial sunscreen and one of three different semiconductor nanocrystalline additives (each at 10 weight percent of the total weight of commercial sunscreen and additive) to demonstrate the effectiveness of the present invention.

FIG. 2 shows a plot of absorption versus wavelength at wavelengths within the UV-A region for several commercial sunscreens and a sunscreen composition based on semiconductor nanocrystals to demonstrate the effectiveness of the present invention.

FIG. 3 shows a plot of absorption versus wavelength at wavelengths within the UV-A region for a commercial sunscreen at various times following sample preparation and for a sunscreen composition based on semiconductor nanocrystals to demonstrate the effectiveness of the present invention. The absorption for the sunscreen composition based on semiconductor nanocrystals remained constant over the time period of the test. The samples were stored under normal fluorescent light.

FIG. 4 shows a plot of absorption versus wavelength at wavelengths within the UV-A and UV-B regions for two different sunscreen compositions based on semiconductor nanocrystals to demonstrate the effectiveness of the present invention, having two different absorption onsets in the visible region of 550 nm and 600 nm, respectively.

FIG. 5 shows a plot of absorption versus wavelength at wavelengths within the UV-A and UV-B regions for: (i) a commercial sunscreen; (ii) a sunscreen composition based on semiconductor nanocrystals to demonstrate the effectiveness of the present invention; and (iii) a blend of the same commercial sunscreen from (i) and the same semiconductor nanocrystals from (ii). Relatively good dispersion of the semiconductor nanocrystals was obtained with this commercial sunscreen.

FIG. 6 shows a plot of absorption versus wavelength at wavelengths within the UV-A and UV-B regions for: (i) another commercial sunscreen; (ii) a sunscreen composition based on semiconductor nanocrystals to demonstrate the effectiveness of the present invention; and (iii) a blend of the same commercial sunscreen from (i) and the same semiconductor nanocrystals from (ii). Relatively poor dispersion of the semiconductor nanocrystals was obtained with this commercial sunscreen.

### DETAILED DESCRIPTION

The present invention is concerned with sunscreen compositions and sunless tanning compositions. Particularly, the present invention is concerned with sunscreen compositions and sunless tanning compositions having substantial broadband absorption properties of ultraviolet light at wavelengths across the range of both UV-A (320-400 nm) and UV-B (280-320 nm). Such compositions are topically applicable. The present invention is further concerned with a process of protecting against detrimental effects of ultraviolet light at wavelengths by applying particular sunscreen compositions.

As used herein, the term “nanocrystals” refers to particles smaller than about 25 nanometers in the smallest dimension or axis, preferably from about about 1 nanometer to about 15 nanometers. The terms are meant to include various other nanostructures such as nanoclusters, nanoshells, nanorods (rod-shaped nanocrystals), nanowires, branched nanorods and the like. Also, within particularly selected nanocrystals, the nanocrystals can be substantially monodisperse, i.e., the particles have substantially identical size and shape. In the case of core/shell structured nanocrystals, the size generally refers to the core of the nanocrystal. For example, if the core of a first material had a 3 nm diameter and was surrounded by a 1 nm thick shell of another material, then the total diameter would total 5 nm.

The nanocrystals are generally members of a crystalline population having a narrow size distribution, although the size distribution can be broadened if desired. Preferably, the nanocrystals used in the present invention are monodisperse, meaning that at least about 80% of the nanocrystals fall within the desired particle size range and the nanocrystals deviate by less than about 10% in root mean squared (rms) diameter, and preferably less than 5%. That is, the nanocrystals have a size distribution with the full width at half maximum (FW) diameter varying by up to ±10 percent. The shape of the nanocrystals can be a sphere, a rod, a disk, a branched structure and the like. The nanocrystals include: a core of a semiconductor nanocrystal or a metallic nanocrystal having a band edge located in the visible range, i.e., from about 400 to 700 nm, or in the infrared range, i.e., from about 700 nm to 1500 nm, such that the nanocrystals can efficiently substantially absorb the UV radiation, i.e., wavelengths below 400 nm.

The sunscreen compositions and/or sunless tanning compositions of the present invention are photostable, i.e., their broadband absorption properties of ultraviolet light at wavelengths across the range of both UV-A (320-400 nm) and UV-B (280-320 nm) do not diminish over time with exposure to the sun. Additionally, sunscreen compositions and/or sunless tanning compositions of the present invention may have better DNA compatibility than wide-bandgap metal oxides such as titanium dioxide and zinc oxide. Also, sunscreen compositions of the present invention can be generated as visibly transparent compositions, although in some formulations such as self-tanning or sunless tanning compositions or make-up compositions, visible transparency may not be the desired outcome.

The sunscreen compositions and/or sunless tanning compositions of the present invention exploit the properties of nanocrystals and in particular nanocrystalline quantum dots. Quantum dots have optical and electronic properties that can be dependent (sometimes strongly dependent) on both the size and the material forming the quantum dots. It is the size range on the order of a few nanometers in which the quantum mechanical characteristics of atoms and molecules often begin to impact and even dominate the classical mechanics of everyday life. In this size range, a material's electronic and optical properties can change and become dependent on size, i.e., the absorption properties are size-tunable. In addition, as the size of a material gets smaller, and therefore more atomic-like, many characteristics change or are enhanced due to a redistribution of oscillator strength and density of states. These effects are referred to as “quantum confinement” effects. For example, quantum confinement effects can cause the energy gap of the nanocrystal quantum dots to increase as the size of the nanocrystal quantum dots decreases or the absorption band edge of the nanocrystal quantum dots to shift to the blue. These quantum confinement effects result in the ability to finely tune many properties of nanocrystal quantum dots, e.g., optical properties, by carefully controlling their size. This control provides a critical aspect in selected sunscreen compositions and/or sunless tanning compositions of the present invention.

By “substantial broadband absorption” is meant that the sunscreen compositions absorb the majority of ultraviolet light at wavelengths across the range of both UV-A (320-400 nm) and UV-B (280-320 nm), preferably at least greater than about 60 percent absorption at all wavelengths across the range of both UV-A (320-400 nm) and UV-B (280-320 nm), and more preferably at least greater than about 80 percent absorption at all wavelengths across the range of both UV-A (320-400 nm) and UV-B (280-320 nm). In some embodiments of the present invention, by selection of the particular ingredients and amounts within the sunscreen compositions, the sunscreen compositions may absorb nearly all of the ultraviolet light (i.e., greater than about 95 percent) at wavelengths across the range of both UV-A (320-400 nm) and UV-B (280-320 nm).

In one aspect of the present invention, the semiconductor nanocrystal is a non-oxide semiconductor nanocrystal or a modified semiconductor nanocrystal. Among suitable non-oxide semiconductor nanocrystals may be included, e.g., a core of the binary formula MX, where M can be cadmium, zinc, mercury, aluminum, iron, lead, tin, gallium, indium, thallium, magnesium, calcium, strontium, barium, copper, and mixtures or alloys thereof and X is sulfur, selenium, tellurium, nitrogen, phosphorus, arsenic, antimony or mixtures thereof; a core of the ternary formula M₁M₂X, where M₁ and M₂ can be cadmium, zinc, mercury, aluminum, iron, lead, tin, gallium, indium, thallium, magnesium, calcium, strontium, barium, copper, and mixtures or alloys thereof and X is sulfur, selenium, tellurium, nitrogen, phosphorus, arsenic, antimony or mixtures thereof; a core of the quaternary formula M₁M₂M₃X, where M₁, M₂ and M₃ can be cadmium, zinc, mercury, aluminum, iron, lead, tin, gallium, indium, thallium, magnesium, calcium, strontium, barium, copper, and mixtures or alloys thereof and X is sulfur, selenium, tellurium, nitrogen, phosphorus, arsenic, antimony or mixtures thereof. Specific examples of suitable non-oxide semiconductor nanocrystal materials can include cadmium selenide (CdSe), cadmium telluride (CdTe), zinc telluride (ZnTe), iron sulfide (FeS), gallium phosphide (GaP), indium phosphide (InP), indium antimonide (InSb) and the like, mixtures of such materials, or any other semiconductor or similar materials. Other useful semiconductor materials may be of silicon carbide (SiC), silver indium sulfide (AgInS₂), silver gallium selenide (AgGaSe₂), copper gallium selenide (CuGaSe₂), copper indium selenide (CuInSe₂), magnesium silicon phosphide (MgSiP₂), zinc silicon phosphide (ZnSiP₂), zinc germanium phosphide (ZnGeP₂) and the like. Still other useful semiconductor materials may be of silicon, germanium, or alpha-tin.

Semiconductor nanocrystals can be conveniently synthesized using colloidal chemical routes such as the solution-based organometallic synthesis approaches for the preparation of CdSe nanocrystals described by Murray et al., J. Am. Chem. Soc., 115, 8706 (1993) or by Peng et al., J. Am. Chem. Soc., 123, 183 (2001), such references incorporated herein by reference. The selection of suitable hydrophobic capping agents versus hydrophilic capping agents will be dependent upon the carrier or medium for the nanocrystal quantum dots.

Silicon nanocrystals can also be prepared in the various manners described by Lie et al., J. of Electroanalytical Chemistry 538-539, 183-190 (2002), by Wilcoxon et al., Phys. Rev. B, 60 (1999) 2704-2714 and by Iwaski et al., J. Appl. Phys., 35 (1996) L551-554.

The core of any non-oxide semiconductor material can have an overcoating on the surface of the core. The overcoating can also be a semiconductor material, such an overcoating having a composition different than the composition of the core. It can also be a metal. The overcoat on the surface of the nanocrystals can include materials selected from among Group II-VI compounds, Group II-V compounds, Group III-VI compounds, Group III-V compounds, Group IV-VI compounds, Group I-III-VI compounds, Group II-IV-V compounds, and Group II-IV-VI compounds. Specific examples of suitable overcoating materials can include cadmium selenide (CdSe), cadmium telluride (CdTe), zinc telluride (ZnTe), iron sulfide (FeS), gallium phosphide (GaP), indium phosphide (InP), indium antimonide (InSb) and the like, mixtures of such materials, or any other semiconductor or similar materials. Other useful semiconductor materials may be of silicon carbide (SiC), silver indium sulfide (AgInS₂), silver gallium selenide (AgGaSe₂), copper gallium selenide (CuGaSe₂), copper indium selenide (CuInSe₂), magnesium silicon phosphide (MgSiP₂), zinc silicon phosphide (ZnSiP₂), zinc germanium phosphide (ZnGeP₂) and the like, mixtures of such materials, or any other semiconductor or similar materials such as silicon, germanium, or alpha-tin. The shell may also be a metal such as gold or the like. The overcoating upon the core material can include a single shell or can include multiple shells. The multiple shells can be of differing materials.

In addition to core/shell structures, the nanocrystals may be a multicomponent nanocrystal such as a semiconductor/semiconductor nanocrystal or may be a hybrid nanocrystal such as a semiconductor/metal nanocrystal. Further, the nanocrystals may have a fused dimer structure, a hetero-rod structure or a hetero-branched structure. One multicomponent structure that may yield suitable results would be a binary composition of TiO₂ and Fe₂O₃ as the iron oxide can effectively quench photocatalytic behavior by the titanium dioxide. The multicomponent nanocrystal may be an alloy or may be a fused dimer where a particle of one material is fused to a particle of a second material.

In another embodiment of the present invention, the nanocrystals can be of a modified semiconductor nanocrystals. Such modified semiconductor nanocrystals can have the absorption edge of their band gap energies red shifted by use of various ligands such as shown by Rajh et al., “Surface Restructuring of Nanoparticles: An Efficient Route for Ligand-Metal Oxide Crosstalk”, J. Phys. Chem. B, 2002, 106(41), 10543-10552 and Rajh et al., “Surface Modification of TiO₂ Nanoparticles with Bidentate Ligands Studied by EPR Spectroscopy”, J. Non-Crystalline Solids, 1996, 205-207, 815-820. Suitable ligands for such modification are those described by Rajh et al., e.g., substituted mercapto-carboxylic acids such as thiolactic acid, or enediol ligands, such descriptions hereby incorporated by reference. By proper modification of the absorption edge of their band gap energies, other semiconductor materials that otherwise might not meet the necessary requirements of broadband absorption properties of ultraviolet light at wavelengths across the range of both UV-A (320-400 nm) and UV-B (280-320 nm) may be used including modified titanium dioxide, modified zinc oxide and modified iron oxide.

In another aspect of the present invention, specific nanocrystal pigments of iron oxide are utilized as the sunscreen agent; the iron oxide nanocrystals having an absorption band gap occurring at wavelengths higher than 400 nm whereby the nanocrystal pigments have the desired substantial broadband absorption properties of ultraviolet light at wavelengths across the range of both UV-A (320-400 nm) and WV-B (280-320 nm). The iron oxide nanocrystals are specifically characterized as having one or more monodispersed size distributions (less than or equal to ±10%), i.e., there will be one or more specific sizes of the nanocrystalline particles. Further, where iron oxide is used as the semiconductor nanocrystals, it is preferred that the nanocrystals are ligand stabilized, e.g., by a fatty acid such as oleic acid, by an amine, a phosphine, a phosphine oxide and the like, or by micelle encapsulation or by a cross-linked polymer shell. Such ligand stabilization can provide solubilization to the nanocrystals via a monolayer of the bound or coordinated ligands. By “solubilization” is meant that the semiconductor nanocrystals or quantum dots avoid aggregation or settling that may lead to opacity or visibility within a sunscreen composition or to streaking within a sunless tanning composition. Such a solubilization is distinct from dispersion. Solubilized semiconductor nanocrystals should yield clear, stable solutions without aggregation or precipitation over time. Further, where iron oxide is used as the semiconductor nanocrystals, it is preferred that such iron semiconductor nanocrystals be monodispersed in size, i.e., the particles have substantially identical size and shape. In that manner, careful control of a desired color palette can be obtained, e.g., particular monodispersions of iron oxide nanocrystals will yield a particular color. Multiple monodispersed portions of differing sizes may be blended to yield the desired color outcome.

In selected sunscreen compositions of the present invention, the metallic nanocrystals can be a group 10 metal or group 11 metal such as platinum, palladium, silver, copper, gold, mixtures thereof and alloys thereof. Alloys may include, e.g., Au/Ag, Au/Cu, Au/Ag/Cu, Au/Pt, Au/Pd, Au/Ag/Cu/Pd and the like. For example, as nanometer-sized alloy particles can exhibit properties distinctly different from the corresponding mono-metal particles, various preparations of multi-metal nanocrystals are known and such particles are sometimes referred to as core-shell bimetallics, partially segregated alloys and pure alloys. By choice of metal such that the surface plasmon resonance band is sufficiently located far into the visible or infrared regions, the metallic nanocrystals can provide substantial broadband adsorption properties of ultraviolet light at wavelengths across the range of both UV-A and UV-B regions. The simple preparation of nanometer-sized monolayer-protected alloy clusters is described by Hostetler et al., “Stable, Monolayer-Protected Metal Alloy Clusters”, J. Am. Chem. Soc., 1998, 120, 9396-9397, such description incorporated herein by reference. Exemplary of suitable gold nanocrystals are those described by Templeton et al., “Monolayer-protected Cluster Molecules”, Acc. Chem. Res. 2000, 33, 27-36, such description incorporated herein by reference.

The metallic nanocrystals may also be of a core/shell structure such as gold nanoshell, e.g., Au on AuS₂, as described by Averitt et al., “Linear Optical Properties of Gold Nanoshells”, J. Opt. Soc. Am. B, 1999, 16, 1824 or Au on SiO₂, as described by Pham et al., “Preparation and Characterization of Gold Nanoshells Coated with Self-Assembled Monolayers”, Langmuir, 2002, 18, 4915-4920. Similarly, the metallic nanocrystals may be in the shape of nanorods such as gold nanorods described by Yu et al., “Gold Nanorods: Electrochemical Synthesis and Optical Properties”, The Journal of Physical Chemistry B, 1997, 101 (34), 6661-6664.

The core of any such metallic nanocrystal material can have an overcoating on the surface of the core. The overcoating can be a suitable ligand such as an alkanethiolate ligand, an arenethiolate ligand, (mercaptopropyl)trimethyloxysilane ligands, thiolated poly(ethylene glycol) ligands and the like. The overcoating can be of a second metal less subject to oxidation where the overcoating is desired to protect the core metal nanocrystals from oxidation. The overcoating may also be a semiconductor material. Any semiconductor overcoat on the surface of the metallic nanocrystals may include materials selected from among Group II-VI compounds, Group II-V compounds, Group III-VI compounds, Group III-V compounds, Group IV-VI compounds, Group I-III-VI compounds, Group II-IV-V compounds, and Group II-IV-VI compounds. Specific examples of suitable overcoating materials can include cadmium selenide (CdSe), cadmium telluride (CdTe), zinc telluride (ZnTe), iron sulfide (FeS), gallium phosphide (GaP), indium phosphide (InP), indium antimonide (InSb) and the like, mixtures of such materials, or any other semiconductor or similar materials. Other useful semiconductor materials may be of silicon carbide (SiC), silver indium sulfide (AgInS₂), silver gallium selenide (AgGaSe₂), copper gallium selenide (CuGaSe₂), copper indium selenide (CuInSe₂), magnesium silicon phosphide (MgSiP₂), zinc silicon phosphide (ZnSiP₂), zinc germanium phosphide (ZnGeP₂) and the like, mixtures of such materials, or any other semiconductor or similar materials such as silicon, germanium, or alpha-tin. The overcoating upon the core metallic nanocrystals may include a single shell or can include multiple shells. The multiple shells may be of differing materials. The overcoating upon the metallic nanocrystals may also be a dielectric material such as glass and the like or the metallic nanocrystals may be overcoated onto a dielectric core.

The nanocrystals used in the sunscreen compositions of the present invention can also be coated with a barrier layer to encapsulate the various materials and keep the material from contact with its surroundings and to minimize the nanocrystals from oxidation. For example, copper nanocrystals have excellent surface plasmon resonance properties, but would fail to provide any significant absorption properties upon oxidation. In other instances, it may be desired to overcome a material to avoid direct contact between the body and the coated material. Suitable barrier layers are well known to those skilled in the art and may include, e.g., a suitable sol-gel, glass or plastic. One suitable example of such a barrier layer is described by Lapidot et al. in U.S. Pat. No. 6,436,375. A suitable barrier layer on a semiconductor core may be a metal such as gold or the like, e.g., a gold layer over a CdSe core.

The sunscreen compositions and/or the sunless tanning compositions of the present invention include an effective amount of the nanocrystals having the substantial broadband absorption properties of ultraviolet light, such an effective amount ranging from about 1 percent by weight to about 50 percent by weight, more preferably from about 1 percent by weight to about 20 percent by weight, and most preferably from about 1 percent by weight to about 10 percent by weight. Compositions including about 10 percent by weight of the nanocrystals have been found especially useful. For artificial tanning compositions, the compositions will generally include above about 10 weight percent.

The nanocrystals in the present invention can have a different color depending upon their precise size due to quantum confinement effects. An aim of the present invention is to utilize quantum confinement effects to tune absoprtion properties of the nanocrystals. For example, nanocrystals of CdSe having a size of about 3.5 nm, yield an orange color, while nanocrystals of CdSe having a size of about 5 nm, yield an red color, and the precise yellow or red tone can be fine-tuned by controlling the particle size and particle size dispersion. Thus, quantum confinement effects coupled with near monodispersity in particle size will permit a breadth in color pallet options that is unique. For formulation of a suitable sunless tanning composition, suitable nanopigments of varying sizes with varying colors may be blended together to yield the desired skin tinting from a sunless tanning composition while also providing the substantial broadband absorption properties of ultraviolet light at wavelengths across the range of both UV-A (320-400 nm) and UV-B (280-320 nm). By selection and modification of the nanocrystals used in the sunscreen compositions of the present invention, optimal absorption properties (to the red side of 400 nm) can be obtained and color effects can be controlled to allow choice of visual appearance.

The sunscreen compositions of the present invention are non-whitening, i.e., non-opaque. They can be applied for effective broadband sunscreen protection without leaving a white appearance upon the skin. In some instances, careful selection of a blend of varying materials and/or sizes of the nanocrystals may yield a fluorescent type glow to the skin. Such an effect may enhance the appearance of the skin as is described in U.S. Published application 2003/0175228, published on Sep. 18, 2003. Compositions of the present invention may be used in a skin treatment product as well.

In the sunscreen compositions and/or sunless tanning compositions of the present invention, the nanocrystals quantum dots can be incorporated into an acceptable carrier adapted for solubilization or dispersion of the nanocrystal quantum dots. By “solubilization” is meant that the nanocrystal quantum dots avoid aggregation or settling that may lead to opacity or a visible precipitate within a sunscreen composition. This is distinct from “dispersion” with respect to the degree of particle stability that is achieved, with solubilization being more akin to true-molecule-like solubility. Such a carrier should be dermatologically acceptable, i.e., the carrier should not have an adverse reaction with the skin upon application, and should be pharmaceutically acceptable, i.e., the carrier should possess acceptable safety characteristics. Typically, the carrier can include a suitable solvent including one or more aqueous or organic solvent, oil or mixture thereof. Examples of suitable solvents include: propylene glycol, polyethylene glycol, polypropylene glycol, polyvinyl pyrrolidine, propylene glycol butyl ether, glycerol, 1,2,4-butanetriol, sorbitol esters, 1,2,6-hexanetriol, ethanol, isopropanol, butanediol, and the like and mixtures thereof. These and other solvents suitable for use in the present invention are described in the C.T.F.A. Cosmetic Ingredient Handbook, 3rd Ed., Cosmetic and Fragrance Assn., Inc., Washington D.C. (1982), incorporated herein by reference.

The sunscreen compositions and/or sunless tanning compositions of the present invention can be prepared according to techniques well known to a person skilled in the art, in particular those intended for the preparation of emulsions of oil-in-water or water-in-oil type. As noted above, selection of suitable hydrophobic capping agents versus hydrophilic capping agents can vary depending upon the carrier or medium for the nanocrystals.

This sunscreen compositions and/or sunless tanning compositions can be provided in particular in the form of a simple or complex emulsion (O/W, W/O, O/W/O or W/O/W), such as a cream or a milk, or in the form of a lotion, a gel or a cream gel, in the form of a powder or of a solid stick and can optionally be packaged in an aerosol and be provided in the form of a foam or a spray.

The sunscreen compositions and/or sunless tanning compositions of the present invention can be provided in the form of a solution that can be applied directly to the skin to protect it against UV radiation. The solution can be applied, e.g., by spraying the solution onto the skin or rubbing the solution onto the skin.

The sunscreen compositions of the present invention may be used for protecting the hair, and may be in the form of a shampoo, an emulsion or a non-ionic vesicle dispersion. Alternatively, the sunscreen compositions may be packaged as an aerosol and may be in the form of a foam or spray. The sunscreen compositions may be a rinse-out composition, to be applied before or after shampooing, before or after dyeing or bleaching or before, during or after permanent-waving or straightening the hair, or the sunscreen compositions may be a composition for permanent-waving, straightening, dyeing or bleaching the hair.

The sunscreen compositions and/or sunless tanning compositions of the present invention may be used as a make-up product. For make-up products such as a conditioning cream, a foundation, a lipstick, an eyeshadow, a face powder, a blusher, a mascara, an eyeliner, a nail polish and the like, the sunscreen compositions may be in liquid, solid or pasty, anhydrous or aqueous form, such as simple or multiple emulsions or alternatively nonionic vesicle dispersions, powders or solid tubes.

The sunscreen compositions and/or sunless tanning compositions of the present invention may also be used to protect items other than keratinous material. The active sunscreen ingredients described herein can be incorporated into suitable film forming polymers for forming protective films upon substrates exposed to sunlight such as wood (e.g., in decks, paneling, flooring, sidings, shingles, moldings and the like), plastics (e.g., in sidings, molding, outdoor furniture and the like), fabrics, and the like. Similarly, the active sunscreen ingredients described herein may be incorporated into eyewear such as glasses and contact lenses to protect the wearer's eyes from UV radiation. Further, the active sunscreen ingredients described herein may be incorporated directly into fabric as opposed to upon a fabric to protect the fabric from UV radiation.

While various oxide materials such as titanium dioxide (TiO₂), zinc oxide (ZnO), and iron oxide (Fe₂O₃) have been previously incorporated into cosmetics and sunscreens, some of them (TiO₂ and ZnO) do not provide protection against the upper UVA range (380-400 nm,) while others (Fe₂O₃) have not been generally recognized as providing substantial broadband sunscreen protection against ultraviolet light at wavelengths across the range of both UV-A (320-400 nm) and UV-B (280-320 nm). One embodiment of the present invention provides a process of protecting against detrimental effects of ultraviolet light at wavelengths across the range of both UV-A (320-400 nm) and UV-B (280-320 nm) by applying a photostable sunscreen composition including: (i) nanocrystals of a material selected from the group consisting of semiconductor nanocrystals, modified semiconductor nanocrystals, multicomponent semiconductor/semiconductor nanocrystals, metallic nanocrystals and hybrid semiconductor/metal nanocrystals, the semiconductor nanocrystals having an absorption band gap occurring at wavelengths higher than 400 nm whereby the nanocrystals have substantial broadband absorption properties of ultraviolet light at wavelengths across the range of both UV-A (320-400 nm) and UV-B (280-320 nm) and the metallic nanocrystals having a surface plasmon resonance occurring sufficiently into the visible or infrared spectral region whereby broad absorption features due to electronic transitions, the broad absorption features located at higher energies, provide substantial broadband absorption properties of ultraviolet light at wavelengths across the range of both UV-A (320-400 nm) and UV-B (280-320 nm); and, (ii) a dermatologically acceptable carrier for the nanocrystals. In the process, the nanocrystals can generally be of the non-oxide semiconductor nanocrystals, modified semiconductor nanocrystals, and metallic nanocrystals described for the compositions of the present invention, but can further includes selected oxide semiconductor nanocrystals such as iron oxide (Fe₂O₃) and copper oxide (Cu₂O). Other oxide materials such as indium oxide (In₂O₃) may be used as well. In a preferred embodiment of the process, the nanocrystals are of silicon or iron oxide.

The present invention is more particularly described in the following examples that are intended as illustrative only, since numerous modifications and variations will be apparent to those skilled in the art.

The sunscreen effectiveness was tested and SPF values were calculated following the procedures described by Diffey et al., J. Soc. Cosmet. Chem., 40, 127-133 (1989), which involve an in vitro method for determining SPF values that correlate well with those obtained from in vivo studies. The key to mimicking in vivo has been to utilize a substrate that mimics human skin. Diffey et al. simply used a piece of Transpore® adhesive tape (3M Company), which has an irregular surface similar to human skin (allowing distribution of topically applied material similar to skin) and the necessary transparency to UV irradiation at wavelengths down to 290 nm.

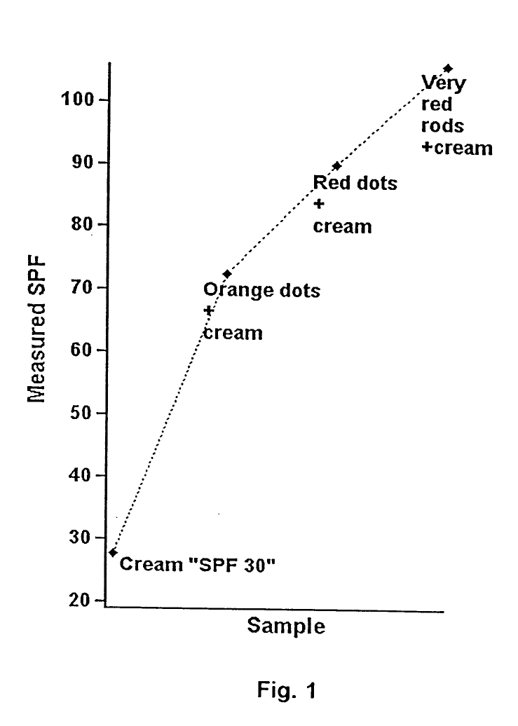

One interesting observation was that SPF values seem to be strongly defined by the UVB region (from about 280 to 320 nm), rather than by the UVA region (from about 320 to 400 nm), or by some more equal representation of each. This is a result of the mathematical formula used to derive the numbers. So, in measuring the SPF values from the nanocrystals of semiconductor nanocrystals, since they offer the greatest difference/improvement with respect to the organic creams in the UVA region, the improvement may not be adequately reflected by SPF numbers. Accordingly, the comparison between the nanocrystals of semiconductor nanocrystals and the commercial sunscreen creams was plotted as absorption, rather than presenting the data simply as SPF values. So, while the experiments were conducted like Diffey et al. and the results demonstrated reasonable SPF values (see FIG. 1), the actual absorption data is shown herein as this may be more informative and meaningful in providing a true comparison across the entire UV spectrum. SPF values shown in FIG. 1 demonstrate enhancement of mixtures with respect to commercial sunscreen creams alone.

A Perkin Elmer UV-vis spectrometer was used for these studies.

Preparation of both red and orange cadmium selenide quantum dots was as described in Murray et al., J. Am. Chem. Soc., 115, 8706 (1993). The cadmium selenide quantum dots were overcoated with a zinc sulfide shell. Preparation of cadmium selenide “frods” was conducted as described by Manna et al., J. Am. Chem. Soc., 122, 12700 (2000).

### EXAMPLE 1

The following commercial sunscreens were tested for absorption (transmission) of light in the UV-A region of the spectrum. The commercial sunscreens included the following: (Perfect Choice® sunscreen having a SPF rating of 4 and including the active ingredients of oxybenzone and ethylhexyl p-methoxycinnamate, available from Inter-American Products, Inc.); (Banana Boat® sunscreen having a SPF rating of 15 and including the active ingredients of octyl methoxycinnamate, oxybenzone and octyl salicylate, available from Sun Pharmaceuticals Corp.); (Coppertone®0 sunscreen having a SPF rating of 15 and including the active ingredients of octyl methoxycinnamate and oxybenzone, available from Schering Plough Health Care Products:); and (Coppertone® sunscreen having a SPF rating of 30 and including the active ingredients of octinoxate, homosalate, oxybenzone, octisalate and avobenzone (Parsol® 1789), available from Schering Plough Health Care Products). One sunscreen composition in accordance with the present invention was formulated by adding semiconductor nanocrystals (CdSe) to the Coppertone® sunscreen having a SPF rating of 30 and tested in the same manner.

Creams were applied onto a substrate (Transpore®0 adhesive tape from 3M, Co.) with a surface area of 3 square inches using a gloved finger. Then substrate was cut in 3 pieces (1 square inch) and 3 separate transmission measurements were made. In measurements involving semiconductor nanocrystals, the semiconductor nanocrystals were first applied onto the substrate using a micropipette and then the commercial sunscreen compositions were applied on top to avoid severe phase separation.

The results shown in FIG. 1 indicate that the combination of cadmium selenide nanocrystals with a commercial sunscreen formulation gave significantly higher SPF values than the commercial sunscreen alone. Also, the tunability of performance by variation in'size of the semiconductor nanocrystals is apparent as the absorption edge is shifted towards the red. The results further demonstrate that the nanocrystal additives do not inhibit activity of commercial organic sunscreen agents and that the present experimental method is valid for replicating known SPF values and, therefore, for evaluating the effectiveness of the nanocrystal additives.

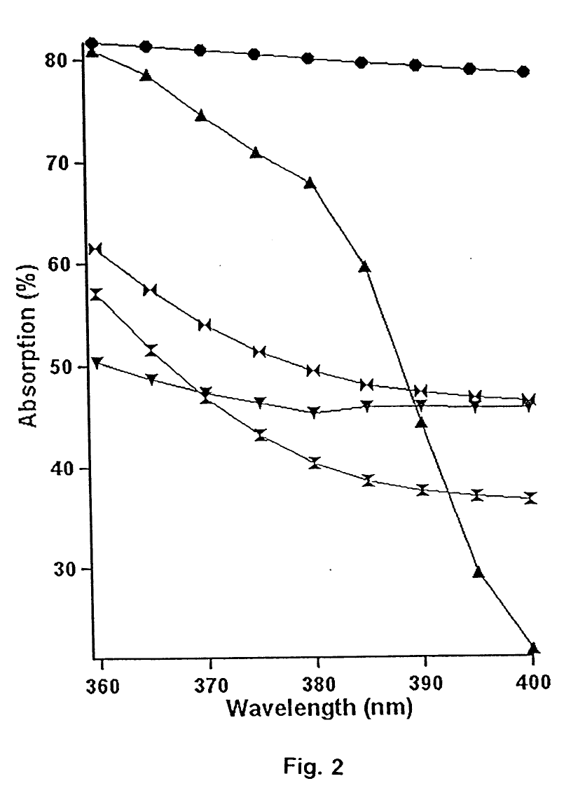

The results shown in FIG. 2 indicate that the commercial sunscreens with the various organic compounds (e.g., oxybenzone, ethylhexyl p-methoxycinnamate, octyl methoxycinnamate, octyl salicylate, octinoxate, homosalate, octisalate and avobenzone) as the active ingredients had a poor absorption (high transmission) within the UV-A region of the spectrum. In contrast, addition of the cadmium selenide nanocrystals to a formulation provided a significantly higher absorption (lower transmission) within the UV-A region of the spectrum. In the FIG. 2, the formulation including the cadmium selenide nanocrystals (the filled circles (●)), was compared to the Coppertone® sunscreen with a SPF rating of 30 (the up facing triangles (▴)), the Coppertone® sunscreen with a SPF rating of 15 (sideways hourglass), the Banana Boat® sunscreen with a SPF rating of 15 (standing hourglass) and the sunscreen with a SPF rating of 4(down facing triangles (▾)) within the wavelength range of 360 to 400 nm.

### EXAMPLE 2

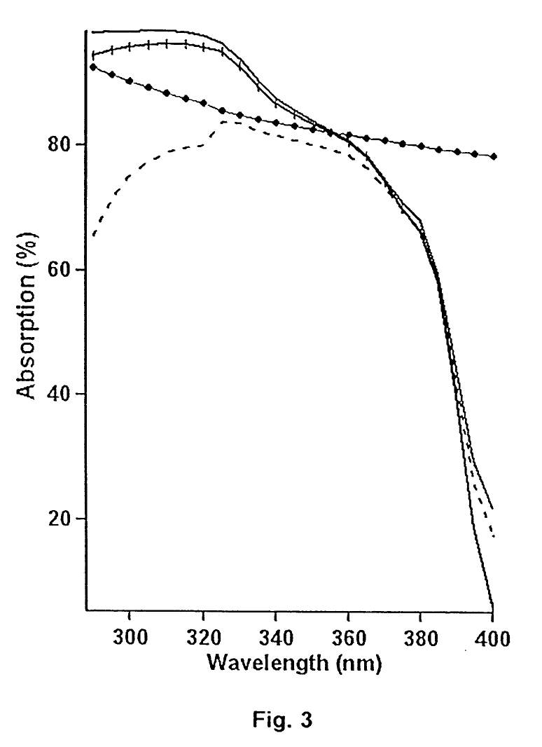

Testing was conducted to measure lifetimes of the sunscreen compositions following exposure to light. In FIG. 3, plots are shown of the cadmium selenide nanocrystals in comparison with the SPF 30 cream for the entire UV spectrum after 0 minutes (line), 2 minutes (slashed line), and 60 minutes (dashed line). The cadmium selenide nanocrystals were spread from a solution in hexane or toluene onto the Transpore® substrate. While it is seen that the high SPF cream has advantage over the cadmium selenide nanocrystals at the concentrations studied at early times (0 and 2 minutes) in the UVB region, it loses this advantage over a one-hour period. There was no change in the performance by the cadmium selenide nanocrystals over time.

The transmission through Coppertone® sunscreen with a SPF rating of 30 was measured immediately after application to the substrate and then re-measured after one hour under white light. The calculated SPF factor dropped from 28.8 to 4.9 during that period.

### EXAMPLE 3

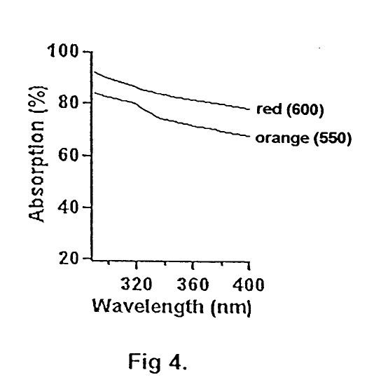

FIG. 4 shows a plot of the absorbance versus wavelength for red cadmium selenide nanocrystals and orange cadmium selenide nanocrystals. The red nanocrystals have an absorption edge beginning at about 600 nm, while the orange nanocrystals have an absorption edge beginning at about 550 nm. These results indicate that by control of the absorption edge onset, the performance in sunscreen protection across both the UVA and UVB can be improved. These results also demonstrate the significance of a fundamental feature of the nanocrystal sunscreen agents, i.e., the position of the optical band gap is key to absorption efficiency in the UV spectral region. The further the band gap is within the visible or infrared spectral region, the more efficient absorption is in the UV; therefore, traditional compositions based on unmodified wide-gap semiconductors, such as TiO₂ and ZnO, are inherently at a disadvantage.

### EXAMPLE 4

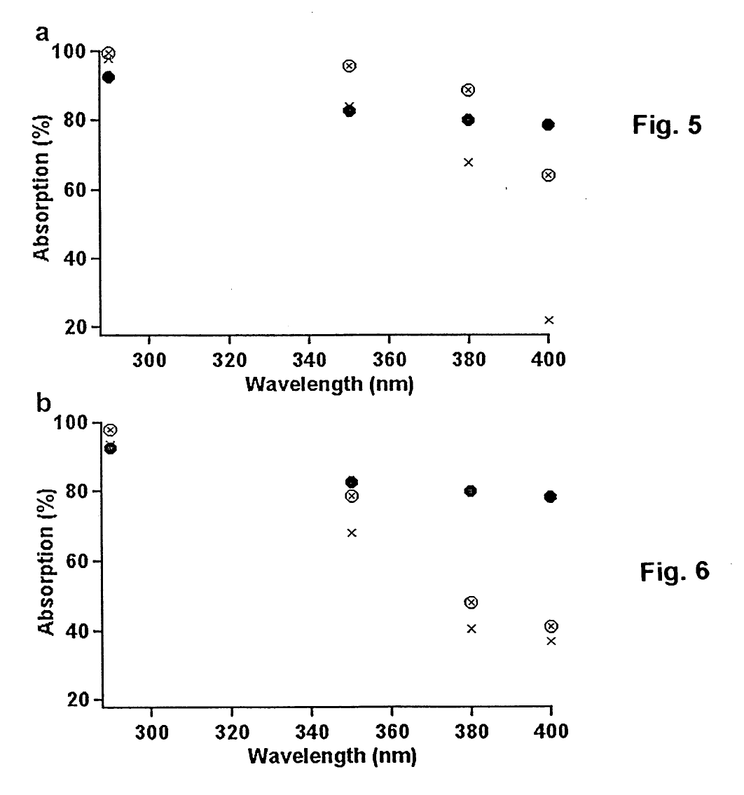

In FIG. 5 and FIG. 6, plots of absorption versus wavelength at wavelengths within the UV-A and UV-B regions are shown comparing performance of dot, cream, and dot/cream combinations at 4 chosen wavelengths. The X symbols are the cream values; solid circle is dots, and X in a circle is the mixtures.

It was found that the mixture (dot/cream) always outperformed the cream, so dot presence does not hinder cream performance. In the run illustrated in FIG. 5, relatively good dispersion was achieved of dots in this cream (cream SPF=30). The mixture outperformed dots alone except at highest wavelength (400 nm), where dots alone were better than mixture, suggesting that dispersion of dots in cream was not ideal and likely resulted in clumping/agglomeration of dots (causing reduced absorption performance of dots in mixtures). In the run illustrated in FIG. 6, poor dispersion of dots in cream was achieved (cream SPF=15). Here, cream/dot mixture outperformed dots alone only for lowest wavelength (280 nm). Otherwise, the dots alone were superior, suggesting that the dots agglomerated significantly in this cream causing reduced performance in the mixture. It should be noted that the amount of dots alone or in mixture was equivalent. It is concluded that the dots as grown (i.e., with a hydrophobic surface) were more compatible with one cream compared to another and this compatibility can strongly affect dot performance as a UV blocking agent. While there were no attempts to optimize the compatibility of the dots to a cream, this is possible by careful choice of ligand-capping agents for the dots.

FIGS. 5 and 6 further demonstrate the importance of additive/carrier compatibility. Incompatibility leads to agglomeration of nanocrystals which reduces absorption efficiency. The nanocrystal quantum dots and the metallic nanocrystals employed in the present invention are unique in comparison to nanoparticles in that they include not only the inorganic core materials, but also a well-defined organic ligand outerlayer, typically approximately a monolayer, but it may be, e.g., a crosslinked polymer shell or a micelle-type encapsulant, that imparts true solubility and tunable solubility that can be tailored precisely to a desired carrier. Though optimization of the compatibility of the nanocrystal additive to a particular cream was not attempted, this is possible through judicious choice of the nanocrystal ligand capping agent. The present invention includes providing for an appropriate nanocrystal capping agent to make the nanocrystal compatible with a particular carrier.

Although the present invention has been described with reference to specific details, it is not intended that such details should be regarded as limitations upon the scope of the invention, except as and to the extent that they are included in the accompanying claims.

1. A photostable sunscreen and/or artificial tanning composition comprising:
an effective amount of quantum dot nanocrystals of a material selected from the group consisting of non-oxide semiconductor nanocrystals, modified semiconductor nanocrystals, multicomponent semiconductor/semiconductor nanocrystals, and hybrid semiconductor/metal nanocrystals, said quantum dot nanocrystals having an absorption band gap occurring at wavelengths higher than 400 nm whereby said quantum dot nanocrystals have substantial broadband absorption properties of ultraviolet light at wavelengths across the range of both UV-A (320-400 nm) and UV-B (280-320 nm); and,
a dermatologically acceptable carrier for said quantum dot nanocrystals.

2. The photostable sunscreen and/or artificial tanning composition of claim 1 wherein said quantum dot nanocrystals in said composition are non-oxide semiconductor nanocrystals.

3. The photostable sunscreen and/or artificial tanning composition of claim 1 wherein said quantum dot nanocrystals in said composition are modified semiconductor nanocrystals.

4. The photostable sunscreen and/or artificial tanning composition of claim 1 wherein said composition further includes metallic nanocrystals.

5. The photostable sunscreen and/or artificial tanning composition of claim 1 wherein said semiconductor nanocrystals are selected from the group consisting of silicon, germanium, alpha-tin, iron sulfide, silicon carbide, gallium phosphide, and indium antimonide.

6. The photostable sunscreen and/or artificial tanning composition of claim 1 wherein said composition is characterized as visibly transparent or translucent.

7. The photostable sunscreen and/or artificial tanning composition of claim 1 wherein said modified semiconductor nanocrystals are from the group selected from ligand-modified titanium dioxide, ligand-modified zinc oxide and ligand-modified iron oxide.

8. The photostable sunscreen and/or artificial tanning composition of claim 1 wherein said semiconductor nanocrystals are of silicon.

9. The photostable sunscreen and/or artificial tanning composition of claim 2 wherein said semiconductor nanocrystals are of silicon.

10. The photostable sunscreen and/or artificial tanning composition of claim 1 wherein said quantum dot nanocrystals of a material selected from the group consisting of non-oxide semiconductor nanocrystals, modified semiconductor nanocrystals, multicomponent semiconductor/semiconductor nanocrystals, or hybrid semiconductor/metal nanocrystals include an overcoat material of another semiconductor material, a modified semiconductor material or a metal.

11. The photostable sunscreen and/or artificial tanning composition of claim 1 wherein said quantum dot nanocrystals are from about 1 to about 25 nanometers in size, and provide sunscreen protection by absorbing the UV light.

12. The photostable sunscreen and/or artificial tanning composition of claim 1 wherein said quantum dot nanocrystals are present at from about 1 to about 50 weight percent of said composition.

13. The photostable sunscreen and/or artificial tanning composition of claim 1 wherein said quantum dot nanocrystals are present at about 10 weight percent of said composition.

14. The photostable sunscreen and/or artificial tanning composition of claim 1 wherein said quantum dot nanocrystals are present in an effective artificial tanning amount of from above about 10 up to about 50 weight percent of said composition, said amount sufficient to yield a tanned appearance.

15. The photostable sunscreen and/or artificial tanning composition of claim 1 wherein said quantum dot nanocrystals of a material selected from the group consisting of non-oxide semiconductor nanocrystals, modified semiconductor nanocrystals, multicomponent semiconductor/semiconductor nanocrystals, and hybrid semiconductor/metal nanocrystals are encapsulated in a sol-gel, a glass or a polymer coating.

16. The photostable sunscreen and/or artificial tanning composition of claim 4 wherein said metallic nanocrystals are selected from the group consisting of gold, silver, platinum, palladium and copper.

17. The photostable sunscreen and/or artificial tanning composition of claim 16 wherein said metallic nanocrystals are encapsulated in a sol-gel, a glass or a polymer coating.

18. A photostable sunscreen and/or artificial tanning composition comprising:
metallic nanocrystals having a surface plasmon resonance occurring sufficiently into the visible or infrared spectral region whereby broad absorption features due to electronic transitions, the broad absorption features located at higher energies, provide substantial broadband absorption properties of ultraviolet light at wavelengths across the range of both UV-A (320-400 nm) and UV-B (280-320 nm); and,
a dermatologically acceptable carrier for said metallic nanocrystals.

19. The photostable sunscreen and/or artificial tanning composition of claim 18 wherein said metallic nanocrystals are from about 1 to about 25 nanometers in size, and provide sunscreen protection by absorbing the UV light.

20. The photostable sunscreen and/or artificial tanning composition of claim 18 wherein said metallic nanocrystals are present at from about 1 to about 30 weight percent of said composition.

21. The photostable sunscreen and/or artificial tanning composition of claim 18 wherein said metallic nanocrystals include a metal shell over a dielectric core so as to provide tunability of the surface plasmon resonance.

22. The photostable sunscreen and/or artificial tanning composition of claim 18 wherein said metallic nanocrystals are encapsulated in a sol-gel, a glass or a polymer coating.

23. The photostable sunscreen and/or artificial tanning composition of claim 18 wherein said composition is characterized as visibly transparent or translucent.

24. The photostable sunscreen and/or artificial tanning composition of claim 18 wherein said metallic nanocrystals are selected from the group consisting of gold, silver, platinum, palladium and copper.

25. A photostable sunscreen and/or artificial tanning composition having broadband absorption properties within the range of both UV-A (320-400 nm) and UV-B (280-320 nm) comprising:
an effective amount of nanocrystal quantum dot pigments of iron oxide, said iron oxide nanocrystal quantum dot pigments, said iron oxide nanocrystal quantum dot pigments having an absorption band gap occurring at wavelengths higher than 400 nm whereby said nanocrystal quantum dot pigments have substantial broadband absorption properties of ultraviolet light at wavelengths across the range of both UV-A (320-400 nm) and UV-B (280-320 nm), said iron oxide nanocrystal quantum dot pigments characterized as having at least one monodispersed size distribution and said iron oxide nanocrystal quantum dot pigments also characterized as soluble by ligand-stabilization of said iron oxide nanocrystal quantum dot pigments; and,
a dermatologically acceptable carrier for said nanocrystal quantum dot pigments.

26. The composition of claim 25 wherein said iron oxide nanocrystals have a single monodispersed size between about 1 nm and 15 nm.

27. The composition of claim 25 wherein said iron oxide nanocrystals include at least two monodispersed sizes between about 1 nm and 15 nm.

28. The composition of claim 25 wherein said iron oxide nanocrystals are ligand stabilized with a layer of a material selected from the group consisting of a fatty acid, an amine, a phosphine and a phosphine oxide.

29. A process of protecting against detrimental effects of ultraviolet light at wavelengths across the range of both UV-A (320-400 nm) and UV-B (280-320 nm) comprising:
applying a photostable sunscreen composition including: (i) nanocrystals of a material selected from the group consisting of semiconductor nanocrystal quantum dots, modified semiconductor nanocrystal quantum dots, metallic nanocrystals, multicomponent semiconductor/semiconductor nanocrystals and hybrid semiconductor/metal nanocrystals, said semiconductor nanocrystals having an absorption band gap occurring at wavelengths higher than 400 nm whereby said semiconductor nanocrystals have substantial broadband absorption properties of ultraviolet light at wavelengths across the range of both UV-A (320-400 nm) and UV-B (280-320 nm) and said metallic nanocrystals having a surface plasmon resonance occurring sufficiently into the visible or infrared spectral region whereby broad absorption features due to electronic transitions, the broad absorption features located at higher energies, provide substantial broadband absorption properties of ultraviolet light at wavelengths across the range of both UV-A (320-400 nm) and UV-B (280-320 nm); and, (ii) a dermatologically acceptable carrier for said nanocrystals.

30. The process of claim 29 wherein said quantum dot nanocrystals are selected from the group consisting of silicon and iron oxide.

31. The process of claim 29 wherein said quantum dot nanocrystals are of silicon.

32. The process of claim 29 wherein said quantum dot nanocrystals are of iron oxide.

33. The process of claim 29 wherein said quantum dot nanocrystals are from about 1 to about 25 nanometers in size, and provide sunscreen protection by absorbing the UV light.

34. The process of claim 29 wherein said quantum dot nanocrystals are present at from about 1 to about 50 weight percent of said composition.

35. The process of claim 29 wherein said quantum dot nanocrystals are present at about 10 weight percent of said composition.

36. The process of claim 29 wherein said quantum dot nanocrystals are encapsulated in a sol-gel, a glass or a polymer coating.
